(19) United States
(12) Patent Application Publication
(10) Pub. No.: US 2022/0404291 A1
(43) Pub. Date: Dec. 22, 2022

(54) BORESCOPE

(71) Applicant: Lufthansa Technik AG, Hamburg (DE)

(72) Inventors: Jan Oke Peters, Hamburg (DE); Michael Thies, Hamburg (DE); Thomas Ruegg, Schaffhausen (CH); Soeren Wedow, Lahnau (DE); Sven Rasche, Hamburg (DE); Jens-Peter Tuppatsch, Hamburg (DE); Soenke Bahr, Darmstadt (DE); Lukas Bath, Hamburg (DE); Thorsten Schueppstuhl, Hamburg (DE); Tarek Mostafa, Schoeffengrund (DE); Oliver Neumann, Solms (DE)

(21) Appl. No.: 17/776,617

(22) PCT Filed: Nov. 13, 2020
(86) PCT No.: PCT/EP2020/082058

§ 371 Date: May 13, 2022

(30) Foreign Application Priority Data
Nov. 15, 2019 (DE) 10 2019 130 949.2

(51) Int. Cl. G01N 21/954; G01N 21/88; G02B 23/24; H04N 5/232

(52) U.S. Cl. CPC G01N 21/954; G01N 21/8806; G02B 23/2484; H04N 5/23238; H04N 2005/2255

(57) ABSTRACT

A borescope includes an electronic image capturing unit having at least one image capturing sensor with a receiving cone at a first end of a shaft, the shaft having a shaft axis and through which data and supply lines for the image capturing unit are led. The image capturing unit is arranged on a rotary head. The rotary head is secured to the first end so as to be rotatable about the shaft axis such that an axis of the receiving cone is not parallel to the shaft axis at the first end and so that the rotating head is arranged to capture a panoramic image.

### CROSS REFERENCE TO RELATED APPLICATIONS

This application is a U.S. National Phase application under 35 U.S.C. § 371 of International Application No. PCT/EP2020/082058, filed on Nov. 13, 2020, and claims benefit to German Patent Application No. DE 10 2019 130 949.2, filed on Nov. 15, 2019. The International Application was published in German on May 20, 2021 as WO 2021/094533 A2 under PCT Article 21(2).

### FIELD

The present disclosure relates to a borescope, in particular for the borescopy of the combustion chambers of aircraft engines, and to an assembly comprising a borescope.

### BACKGROUND

Borescopes can be used for the inspection of industrial devices in areas which are not immediately visible. The borescopes can be inserted into the areas in question through small openings and, either directly or via an optical unit or via a display of a video image captured by suitable sensors on the borescope tip—also called a video borescope—offer an insight into areas that are otherwise not visible.

Borescopy is used, for example, during the inspection of aircraft engines, in order to obtain an insight into the interior of the engine, without having to take it apart with a great deal of effort for the purpose. Here, at least for individual areas of the aircraft engine, such as, for example, the combustion chamber, it is required or at least desirable to analyze and to document the area completely.

At the current time, for the borescopy of the interior of the combustion chamber, use is made of a video borescope with a flexible shaft, which is guided manually through the combustion chamber. For this purpose, the flexible borescope is guided along the complete inner circumference of the combustion chamber and then drawn out slowly. During the withdrawal, the images captured by the borescope are recorded. In the process, it is attempted to ensure that the complete circumference of the usually ring-shaped combustion chamber is captured. If a possible problem location in the combustion chamber is identified, manual three-dimension (3D) capture of the corresponding point with separate 3D borescopes that are suitable for the purpose can then be carried out.

Because of the manual guidance of the borescope with a flexible shaft, the inventors have recognized that complete and reproducible documentation of the condition of a combustion chamber is, however, barely possible. In addition, the inventors have recognized that, in particular the subsequent 3D capture of possible problem locations is very complicated and time-consuming.

### SUMMARY

In an embodiment, the present disclosure provides a borescope that includes an electronic image capturing unit having at least one image capturing sensor with a receiving cone at a first end of a shaft, the shaft having a shaft axis and through which data and supply lines for the image capturing unit are led. The image capturing unit is arranged on a rotary head. The rotary head is secured to the first end so as to be rotatable about the shaft axis such that an axis of the receiving cone is not parallel to the shaft axis at the first end and so that the rotating head is arranged to capture a panoramic image.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 shows a schematic illustration of the borescope tip of an exemplary embodiment of the borescope according to the invention;

FIG. 2 shows a schematic illustration of the borescope tip of an exemplary embodiment of a borescope according to the invention; and

FIG. 3 shows a schematic illustration of an assembly according to the invention comprising a borescope according to FIG. 1 or 2.

### DETAILED DESCRIPTION

Aspects of the present disclosure provide a borescope with which the inspection of industrial devices, in particular of the combustion chambers of aircraft engines, can be simplified and improved.

Accordingly, an embodiment of the present disclosure relates to a borescope, in particular for the borescopy of the combustion chambers of aircraft engines, comprising an electronic image capturing unit with at least one image capturing sensor with a receiving cone at a first end of a shaft which has a shaft axis and through which data and supply lines for the image capturing unit are led, wherein the image capturing unit is arranged on a rotary head which is secured to the first end so as to be rotatable about the shaft axis such that the axis of the receiving cone is not parallel to the shaft axis at the first end and a panoramic image can be captured by rotating the rotary head.

Furthermore, an embodiment of the present disclosure relates to an assembly comprising a borescope as claimed in one of the preceding claims and a control and evaluation unit, which is designed to control the rotational movement of the rotary head and the image capturing unit and to combine the image data captured by the at least one image capturing sensor into a panoramic image.

The inventors have recognized that, for the borescopy of industrial devices, in particular the combustion chambers of aircraft engines, it is advantageous if the borescope used is designed to create panoramic images—i.e. a 360° panoramic image. Once the borescope has been moved to a desired position, according to an aspect of the present disclosure, the panoramic image can be created without changing the position or location of the borescope shaft.

To this end, provision is made for the at least one image sensor of the image capturing unit to be arranged on a rotary head which can be rotated about the shaft axis. Here “shaft axis” designates the longitudinal axis or axis of symmetry of the shaft. If the shaft axis does not extend linearly (for example in the case of a curved shaft) and/or if it is variable (for example in the case of a flexible shaft), the focus is on that part of the shaft axis immediately at the first end of the shaft on which the rotary head is arranged as an axis of rotation for the rotary head.

The rotary range of the rotary head can be less than or equal to 360°. By means of an appropriate limitation of the rotary range, it is possible to prevent the data and supply lines possibly led out of the shaft as far as the rotary head from twisting or winding up during any rotation of the rotary head. Since it is simultaneously sufficient for the creation of a panoramic image if the entire 360° range is actually captured by the receiving cone of the image capturing unit, a rotary range of less than 360° may also be sufficient, since the receiving cone regularly has an extent in the plane perpendicular to the axis of rotation of the rotary head, so that a complete panoramic image can nevertheless be created.

The rotary head preferably has an internal gear, in which a pinion driven by a drive unit secured so as to be stationary and eccentric with respect to the shaft axis engages. As a result of the eccentric arrangement of the drive unit with respect to the shaft, the guidance of the data and supply lines on the shaft into the rotary head can be simplified. The drive unit can be an electric motor, preferably a stepper motor, the supply and control lines of which can likewise be led through the shaft.

The rotary head preferably comprises a co-rotating cylindrical housing having at least one transparent window, in which the image capturing unit is arranged in such a way that the receiving cone of each image capturing sensor is respectively aimed through a transparent window. The image capturing unit is protected by the housing while, because of the co-rotating window provided therein, no restriction is to be expected with regard to the image capture at any desired angular positions of the rotary head.

Alternatively, a cylindrical housing that is stationary at the first end with respect to the shaft axis, surrounds the rotary head and has at least one transparent ring segment can be provided, wherein the receiving cone of each image capturing unit is respectively aimed through a transparent ring segment, wherein, for each individual image capturing sensor (respectively) a separate ring segment can be provided and/or the receiving cone of a plurality of image capturing sensors can be aimed through a common ring segment. In this case, although the housing is stationary, because of the at least one ring segment, the image capture by the image capturing sensor is not impaired in any angular position of the rotary head.

In both cases, the housing has a cylindrical shape. The housing can thus be viewed as a rigid continuation of the shaft, with which in particular the insertion of the borescope according to an embodiment of the present disclosure into a borescope opening is simply possible. The external diameter of the housing can preferably correspond approximately to the external diameter of the shaft.

The housing—in both the aforementioned embodiments—is preferably encapsulated in a liquid-tight manner. The borescope can also be used for liquid-filled cavities without the image capturing unit or other components of the borescope in the area of its tip coming into direct contact with the liquid and being damaged as a result.

It is preferred for the image capturing unit to comprise at least two image capturing sensors spaced apart from one another, preferably in the direction of the shaft axis, having receiving cones at least partly intersecting and/or aimed parallel to each other for determining 3D information by means of triangulation. Since the two image capturing sensors of the pair spaced apart from each other capture a common image section, with the aid of triangulation it is possible to determine 3D information about the spacing of the image points received by the two image capturing sensors, which can be combined later to form a 3D model of the borescoped area. Suitable triangulation methods are known from the prior art.

It is preferred for the image capturing sensors of a pair provided for the triangulation to be arranged with a center spacing of 15 mm to 25 mm, preferably of 17 mm to 22 mm, more preferably of about 20 mm. Alternatively, a center spacing of 5 mm to 15 mm, preferably 7 mm to 12 mm, more preferably 10 mm to 11 mm is preferred. “Center spacing” designates the spacing of the two sensor centers relative to each other. The accuracy of the determination of the 3D data with the aid of triangulation depends on the spacing of the two image capturing units, the limited available installation space and optical distortions because of the regularly only small spacing of the capture plane from the image capturing unit being limiting factors. The aforementioned spacings have proved to be advantageous in particular for the use of the borescope according to an embodiment of the present disclosure for the inspection of aircraft engines.

The image capturing sensors can be arranged and/or configured in such a way that the receiving cone of one or two image capturing sensors provided for the capture of 3D information is/are arranged with a predefined viewing angle to the longitudinal axis of the image capturing unit. If this viewing angle is 90°, areas at the side of the image capturing unit can be captured. By means of a different selection of the viewing angle differing from 90°, areas located in front in the insertion direction of the borescope (angular range of 30°-90°)or areas located further back (angular range 90°-150° can be captured. However, it is also possible to provide a plurality of image capturing sensors or pairs of image capturing sensors provided for triangulation, which each have different viewing angles, on a single borescope. In particular, two pairs of image capturing sensors can be provided, wherein the receiving cones of the two image capturing sensors of one pair can be aimed at a different viewing angle with respect to the shaft axis than the receiving cones of the two image capturing sensors of the other pair.

The image capturing unit can comprise at least one image capturing sensor for capturing color images. The color images captured by this at least one image capturing sensor can be used directly as a panoramic image. However, it is also possible for an item of 3D information determined on the basis of gray-value images captured by one pair of image capturing sensors to be supplemented with the color information from a color image capturing sensor, in order thus to obtain color 3D information or a color 3D model. The use of gray-value image capturing sensors for determining 3D information may be advantageous because of the higher resolution as compared with color image capturing sensors of an identical sensor size.

The image capturing sensors are preferably CCD sensors or CMOS sensors, preferably with a global shutter. The image capturing sensors preferably have a resolution of 400×400 pixels to 2400×2400 pixels, an image repetition rate of up to 240 frames per second and/or an angle of field of 30° to 120°, preferably 35° to 65°, more preferably of 40°, 50° or 60°, in each case ±5°, preferably in each case ±3°. By using appropriate image capturing sensors, continuous capture of image information is in particular also possible.

It is preferred if at least one light source, preferably an LED, is arranged on the rotary head to illuminate the capture area. As a result of arranging the light source directly on the rotary head, good illumination and lighting of the capture area can be ensured, irrespective of the angular position of the rotary head. The at least one light source can emit visible light and/or infrared radiation, depending on the wavelength range for which the image capturing sensors are designed. It is of course also possible to provide a plurality of different light sources, for example one for the visible and one for the infrared range. The use of LEDs as light sources is particularly preferred because of the low development of heat and the low energy consumption.

The shaft of the borescope can be rigid, semi-flexible or flexible. If the shaft is flexible, the borescope can, for example, be led through a guide tube. The guide tube can be part of the borescope or of a separate guide device. Via the guide tube, the fundamental position of the borescope or its image capturing unit in the interior of the area to be borescoped can be defined. The shaft can also be provided with control cables, which permit control of the shaft. However, it is also possible to guide the borescope having a flexible shaft loosely through an area to be recorded and to create the desired recordings in particular during the withdrawal of the borescope.

In the assembly according to an embodiment of the present disclosure, a control and evaluation unit connected to the borescope according to an embodiment of the present disclosure is provided, with which the rotational movement of the rotary head and the at least one image capturing unit is controlled and with which the individual images captured by the at least one image capturing sensor can be combined into a panoramic image.

The assembly can be designed for continuous capture by the image capturing sensors during rotation of the rotary head. In other words, in a short sequence—as a rule predefined only by the speed of the image capturing sensors—images are captured as the rotary head rotates. Appropriate continuous capture permits a high quality in the panoramic image assembled on the basis of these images.

Alternatively, it is possible that the assembly is designed to capture individual images by the image capturing unit at angular positions reached one after another during rotation of the rotary head. The angular positions should be chosen such that the individual images can continue to be combined into a panoramic image. As compared with continuous capture by the image capturing sensors, the quantity of data to be processed is smaller in this alternative.

The control and evaluation unit is preferably designed to combine two partly overlapping panoramic images. By combining overlapping panoramic images, an enlarged panoramic image can be created. The control and evaluation unit can also be used to control the change in the position of the rotary head, from each of which a panoramic image is to be captured. Suitable controllable guide devices for this purpose are known in the prior art.

The combining of individual images into panoramic images or of individual panoramic images into an enlarged panoramic image comprises the combining of the associated 3D information, if this has been determined by the borescope or by the control and evaluation unit. In this way, a 3D model of the borescoped area is produced.

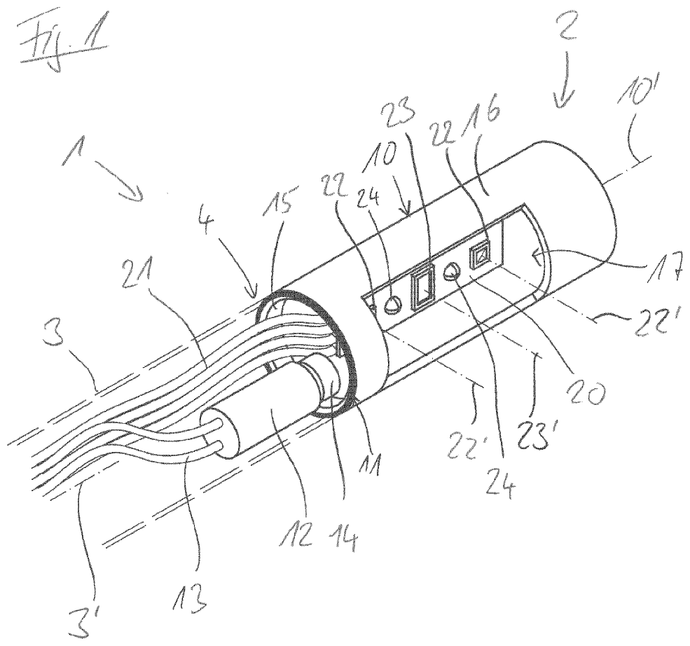

FIG. 1 shows, schematically, the tip 2 of a borescope 1, which tip is inserted into the areas to be examined. The borescope 1 comprises a flexible shaft 3, controllable via control cables. At the first end 4 of the shaft 2, close to the tip, there is arranged a rotary head 10, which is mounted via a bearing 11 such that it can rotate about the shaft axis 3′. The shaft axis 3′ is designated the axis of symmetry of the shaft 3, wherein the axis of rotation 10′ of the rotary head 10 coincides with the shaft axis 3′ directly at the first end 4 of the shaft 3, so that the remaining instantaneous shape of the flexible shaft 3 does not matter. If the shaft axis 3′ is mentioned below, the part of the shaft axis 3′ directly adjacent to the first end 4 of the shaft 3 is meant.

On the bearing 11, a stepper motor is secured in a fixed location with respect to the shaft 3 and its shaft axis 3′ as a drive unit 12. The drive unit 12 is arranged eccentrically relative to the shaft 3, so that sufficient space remains for data and supply lines 21 to be led through from the shaft 3 into the rotary head 10. The drive unit 12 is connected to control and supply cables 13, which are likewise led through the shaft 3 and via which the drive unit 12 can be controlled.

The drive unit 12 engages with a pinion 14 in an internal gear 15 on the rotary head 10, and can thus rotate the rotary head 10 about its axis of rotation 10′ and the shaft axis 3′. The range of rotation of the rotary head 10 is limited by suitable stops to about 280°, in order to prevent the data and supply lines 21 butting against the possibly heat-developing drive unit 12 or twisting.

The rotary head 10 comprises a co-rotating cylindrical housing 16 with a transparent window 17. The housing 16 is encapsulated in a liquid-tight manner.

Arranged in the interior of the rotary head 10 or its housing 16 is an image capturing unit 20, which is attached to the data and supply lines 21.

The image capturing unit 20 comprises two gray-value image capturing sensors 22 which are spaced apart from one another and the receiving cones of which intersect in such a way that 3D information for the overlapping area can be derived from the images from the two image capturing sensors 22 by triangulation. Furthermore, a color image capturing sensor 23 is provided, which likewise captures the overlapping area of the two other image capturing sensors 22. The color image information from the image capturing sensor 23 can be used to enhance the 3D information obtained via the two other image capturing sensors 22 with color information. Appropriate methods for this purpose are known in the prior art.

The image capturing unit 20 also comprises two LEDs as light sources 24, with which the capture area of the individual image capturing sensors 22, 23 can be adequately lit.

The image capturing unit 20 is arranged within the housing 16 of the rotary head 10 such that both the image capturing sensors 22, 23 capture the surroundings through the transparent window 17, and also the light sources 24 can illuminate the surroundings through the transparent window 17.

The image capturing sensors 22, 23 are also arranged such that their receiving cones or their receiving axes 22′, 23′ are oriented at a predefined viewing angle of 90° with respect to the shaft axis 3′ and the axis of rotation 10′.

Since the image capturing unit 20 is fixed in its location with respect to the housing 16 and is thus rotatable about the axis of rotation 10′ by 280°, the result, together with the receiving areas of the image capturing sensors 22, 23, is the possibility of an annular 360° panorama solely as a result of rotation of the rotary head 10. The image data and 3D information captured by the image capturing sensors 22, 23 can be combined appropriately into a panoramic image.

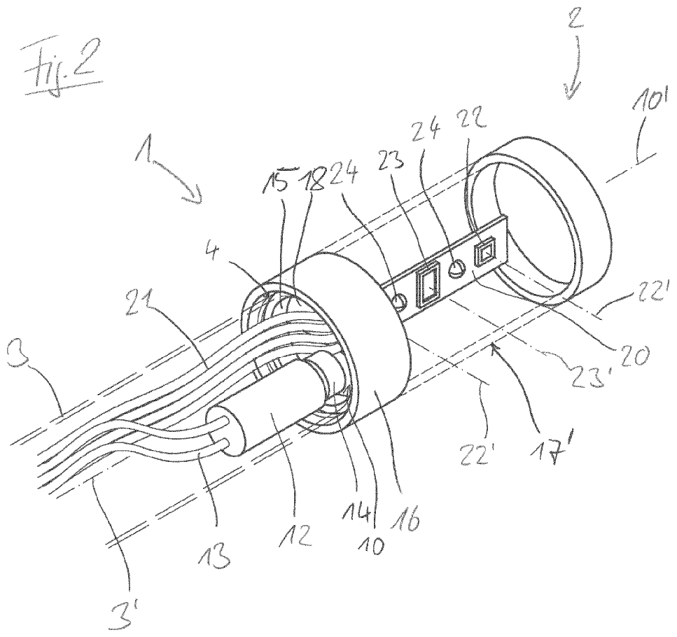

In FIG. 2, an exemplary embodiment of a borescope 1 is shown, wherein there is broad agreement with the exemplary embodiment from FIG. 1. In the following, only the differences of the alternative exemplary embodiment will therefore be discussed and otherwise reference will be made to the above explanations.

In the exemplary embodiment according to FIG. 2, the housing 16 is designed to be fixed with respect to the shaft 3, and the parts of the rotary head 10 that are rotatable about the axis of rotation 10′ comprise the image capturing unit 20 already fixed to a holder 18 of the internal gear 15 within the housing 16. The non-visible bearing is provided between the internal gear 15 and the inner wall of the housing 16. The first end 4 of the shaft 3 is inserted into the housing 16 and firmly connected thereto.

In order that the image capturing sensors 22, 23 of the image capturing unit 20 projecting from the holder 18 can capture the surroundings in every angular position to which the drive unit 12 can be driven, the housing has a completely transparent ring segment 17′. The ring segment 17′ is connected to the remaining non-transparent parts of the housing 16 in such a way that the housing 16 is liquid-tight as a whole; the rotary head 10 is therefore encapsulated in a liquid-tight manner.

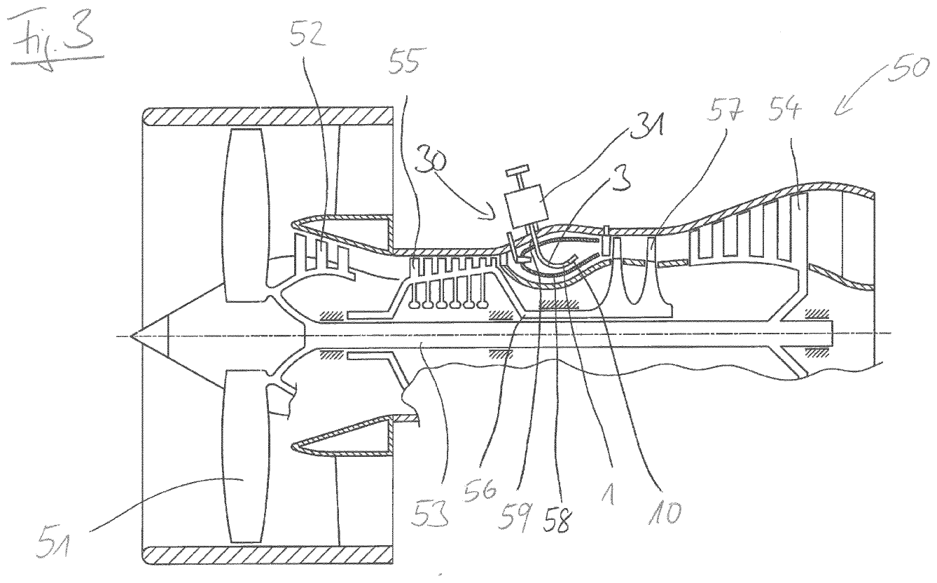

FIG. 3 shows, schematically, a section through a two-shaft engine 50 in which the fan 51 and the low-pressure compressor 52 are rotationally connected via a first shaft 53 to the low-pressure turbine 54, while the high-pressure compressor 55 is rotationally connected via a second shaft 56 to the high-pressure turbine 57. The annular combustion chamber 58 is arranged between the high-pressure compressor 55 and the high-pressure turbine 57.

In addition to a borescope 1, which is designed according to one of FIG. 1 or 2 and consequently comprises a rotary head 10, the assembly 30 comprises a control and evaluation unit 31. Since the control and evaluation unit 31 also comprises the actuators for the control cables of the controllable shaft 3, it is secured directly to the engine 50 in the area of a borescope opening 59, through which the borescope 1 is inserted into the combustion chamber 58.

The control and evaluation unit 31 is connected to the image capturing unit 20 and the drive unit 12 via the data, control and supply lines 14, 21 running in the shaft 3 of the borescope 1 (cf. FIGS. 1 and 2). Since the control and evaluation unit 31 can, moreover, control the shaft 3 via its control cables, completely automatic 3D capture of the combustion chamber 58 is possible.

For this purpose, the control and evaluation unit 31 controls the control cables of the shaft 3 such that predefined positions within the combustion chamber 58 can be approached by the rotary head 10 one after another. At each of these positions, by rotating the rotary head 10 and simultaneously capturing the surroundings by means of the image sensors 22, 23, 3D information and color information is then collected, which is then combined by the control and evaluation unit 31 into color 3D panoramic images by using known triangulation and sampling methods. The imaging sensors 22, 23 are able to capture images continuously as the rotary head 10 rotates, or individual images are captured only at specific angular positions of the rotary head 10. In both cases, the image information can be combined into color panoramic images comprising 3D information.

The overlapping color 3D panoramic images captured at the various points can then be combined further into a 3D model of the interior of the combustion chamber 58, which can then be assessed and analyzed at a user terminal.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A borescope, the borescope comprising:
an electronic image capturing unit comprising at least one image capturing sensor with a receiving cone at a first end of a shaft, the shaft having a shaft axis and through which data and supply lines for the image capturing unit are led,
wherein the image capturing unit is arranged on a rotary head, the rotary head being secured to the first end so as to be rotatable about the shaft axis such that an axis of the receiving cone is not parallel to the shaft axis at the first end and so that the rotating head is arranged to capture a panoramic image.

2. The borescope as claimed in claim 1, wherein a rotary range of the rotary head is less than or equal to 360°.

3. The borescope as claimed in claim 1, wherein the rotary head has an internal gear, in which a pinion engages, the pinion being driven by a drive unit fixed so as to be stationary and eccentric with respect to the shaft axis.

4. The borescope as claimed in claim 1, wherein the rotary head comprises a co-rotating cylindrical housing having at least one transparent window, in which the image capturing unit is arranged in such a way that the receiving cone of each of the at least one image capturing sensor is respectively aimed through the corresponding at least one transparent window.

5. The borescope as claimed in claim 1, the borescope comprising a cylindrical housing, which is stationary at the first end with respect to the shaft axis, surrounds the rotary head and has at least one transparent ring segment, wherein the receiving cone of the image capturing unit is respectively aimed through the corresponding at least one transparent ring segment.

6. The borescope as claimed in claim 4, wherein the cylindrical housing is encapsulated in a liquid-tight manner.

7. The borescope as claimed in claim 1, wherein the at least one image capturing sensor of the image capturing unit comprises at least two image capturing sensors spaced apart from one another, having receiving cones at least partly intersecting or aimed parallel to each other for determining three-dimension (3D) information by triangulation.

8. The borescope as claimed in claim 1, wherein the at least one image capturing sensor is arranged or configured such that the receiving cone of the corresponding at least one image capturing sensor or of each of a pair of image capturing sensors, comprised by the at least one image capturing sensor, provided for the capture of three-dimension (3D) information is oriented at a predefined viewing angle with respect to the shaft axis at the first end.

9. The borescope as claimed in claim 1, wherein the image capturing unit and the at least one image capturing sensor are designed to capture color images.

10. The borescope as claimed in claim 1, wherein at least one light source is provided on the rotary head to illuminate the capture area.

11. The borescope as claimed in claim 1, wherein the shaft is configured as a flexible shaft.

12. An assembly comprising the borescope as claimed in claim 1 and a controller that is configured to control the rotational movement of the rotary head and the image capturing unit and to combine the image data captured by the at least one image capturing sensor into the panoramic image.

13. The assembly as claimed in claim 12, wherein the assembly is designed for continuous capture by the image capturing unit during rotation of the rotary head.

14. The assembly as claimed in claim 12, wherein the assembly is designed to capture individual images by the image capturing unit at angular positions reached one after another by rotation of the rotary head.

15. The assembly as claimed in claim 12, wherein the controller is configured to combine two partly overlapping panoramic images into the panoramic image.
